(19) United States
(12) Patent Application Publication
(10) Pub. No.: US 2018/0197629 A1
(43) Pub. Date: Jul. 12, 2018

(54) SMART TRACKING OF BABY FEEDING HABITS

(71) Applicant: BlueSmart Technology Corporation, Mountain View, CA (US)

(72) Inventor: Dengping Zhou, Hubei (CN)

(21) Appl. No.: 15/740,731

(22) PCT Filed: Jul. 1, 2015
(86) PCT No.: PCT/CN2015/083103

§ 371 Date: Dec. 28, 2017

(51) Int. Cl. G16H 20/60; A61J 9/00

(52) U.S. Cl. CPC G16H 20/60; A61B 2503/04; A61J 9/00

(57) ABSTRACT

A solution is provided for monitoring and analyzing baby feeding habits anywhere and anytime. The baby feeding habits data, e.g., feeding times, types of content consumed (milk, water, juice or baby formula) and volume of intakes, are collected through a variety of electronic sensors, e.g., a temperature sensor, a motion sensor, a color sensor and an electronic field imaging sensor, attached to a baby feeding bottle. The baby feeding habits data are recorded and analyzed in real time. The baby feeding habits data associated with other babies are correlated among each other and with benchmark data. The analysis results and nutritional recommendations are presented in a user friendly way to users on their computers and smart phones.

### BACKGROUND

This invention relates generally to digital content processing and particularly to monitoring baby feeding habits data through electronic sensors and analyzing the baby feeding habits data.

It is generally known that poor eating habits can lead to long-term health issues such as diabetes. Problems of obesity and inappropriate nutrition are increasingly alarming in the field of public health. For example, children and even infants are showing signs of over-eating in increasing numbers. One of the possible reasons of infants suffering from inappropriate nutrition is that their nutrition intake is either inadequate or excessive or their choice of foods fails to provide sufficient amount of various essential nutritional needs. Monitoring baby feeding habits, e.g., amount of nutrition intakes, enables a deeper understanding of children's nutritional habits and recommendations for healthy nutritional habits can be made by doctors.

One existing solution is to manually record an infant's feeding amount and characteristics of the infant, e.g., weight and activity, at each feeding. The manually recorded feeding data is analyzed and used to calculate recommended quantities of consumption. However, manual recording and monitoring of baby feeding habits are tedious and prone to errors; this is especially challenging when infants need to be constantly fed.

On the other hand, smart handheld devices, such as smart phones and tablet computers, have become increasingly popular. The increased availability and bandwidth of network access (for wired and wireless networks) have enabled more communication platforms for digital content consumption and sharing, such as remotely monitoring electronic monitoring devices by smart phones and sharing information with others on social networking platforms. Thus, manually recording and monitoring baby feeding habits without effectively making use of the advancement of mobile technologies is hard to be efficient and effective.

### SUMMARY

Embodiments of the invention provide a solution for monitoring and analyzing baby feeding habits anywhere and anytime. The baby feeding habits data, e.g., feeding times, types of content consumed (milk, water, juice or baby formula) and volume of intakes, are collected through a variety of electronic sensors attached to a baby feeding bottle. The baby feeding habits data are recorded and analyzed in real time. The analysis results and nutritional recommendations are presented in a user friendly way to users on their computers and smart phones.

One aspect of the invention provides a monitoring device attached to a baby feeding bottle for monitoring baby feeding habits. The monitoring device is safely charged by a wireless charger. The monitoring device use a variety of electronic sensors, e.g., a temperature sensor, a motion sensor, a color sensor and an electronic field imaging sensor, to collect sensor data describing various aspects of the baby feeding habits. For example, the temperature sensor detects the temperature of the baby feeding bottle; the motion sensor detects the angle of the baby feeding bottle; the color sensor detects the color of the content in the baby feeding bottle; and the electronic field imaging sensor detects the proximity of the content in the baby feeding bottle. The monitoring device provides the collected baby feeding habits data to a computer server for real time and in-depth analysis. If the analysis of the collected baby feeding habits data, e.g., calculating the volume of the content in the baby feeding bottle, is light weight (e.g., it does not require a large amount of computing power), the monitoring device provides the real time analysis to users of a baby feeding monitoring software application on their computers and smart phones.

Another aspect of the invention provides a computer server for recording and analyzing baby feeding habits data in real time. The computer server receives the baby feeding habits data collected from many monitoring devices supported by the computer server. For each set of baby feeding habits data associated with a baby, the computer server calculates the volume of content in the baby feeding bottle and other statistics associated with the received baby feeding habits data. The baby feeding habits data associated with multiple babies are correlated among each other and with benchmark data. The analysis results and nutritional recommendations are presented in a user friendly way to users on their computers and smart phones.

The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes, and may not have been selected to delineate or circumscribe the disclosed subject matter

### BRIEF DESCRIPTION OF THE DRAWINGS

FIG. 1 is a block diagram of a computing environment for monitoring baby feeding habits and for analyzing baby feeding habits data according to one embodiment.

FIG. 2 is a block diagram illustrating an example of a computer for acting as a client device and/or a computer server to monitor baby feeding habits and to analyze baby feeding habits data according to one embodiment.

FIG. 3 is a block diagram of a sensor module to collect baby feeding habits data through various electronic sensors according to one embodiment.

FIG. 4 is a block diagram of a control module to determine volume data of an eatable content contained in a baby feeding bottle according to one embodiment.

FIG. 5 is a block diagram of an interface module of a computer server to communicate with client devices and monitoring devices according to one embodiment.

FIG. 6 is a block diagram of an analysis module to analyze baby feeding habits data according to one embodiment.

FIG. 7 is an exemplary flowchart illustrating a process for analyzing baby feeding habits data by a computer server according to one embodiment.

FIG. 8 is an exemplary flowchart illustrating a process for monitoring baby feeding habits data through various electronic sensors according to one embodiment.

FIG. 9 illustrates examples of graphical user interface to present various types of information associated with baby feeding habits data on a client device according to one embodiment.

FIG. 10 illustrates examples of a graphical representation of a dashboard for presenting baby feeding habits data on a client device according to one embodiment.

FIG. 11 is an exemplary graphical representation of a dashboard showing a daily baby feeding habits data on a client device according to one embodiment.

FIG. 12 illustrates an exemplary graphical user interface to present historical baby feeding habits data at a client device according to one embodiment.

FIG. 13 illustrates an exemplary graphical user interface of a baby profile shown at a client device according to one embodiment.

FIG. 14 illustrates an exemplary graphical user interface to present feeding scheduling information at a client device according to one embodiment.

FIG. 15 illustrates an exemplary graphical user interface to display baby feeding status and alerts at a client device according to one embodiment.

FIG. 16 illustrates an example of a baby feeding bottle and a monitoring device attached to the baby feeding bottle according to one embodiment.

FIG. 17 is an exemplary monitoring device attached to a baby feeding bottle to collect baby feeding habits data according to one embodiment.

FIG. 18 is an exemplary wireless charger to charge a monitoring device attached to a baby feeding bottle according to one embodiment.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### System Overview

A solution is provided to monitor and to analyze baby feeding habits anywhere and anytime through a variety of electronic sensors attached to a baby feeding bottle. The analysis, nutritional recommendations and predictions are presented in a user friendly way to users on their computers and/or smart phones. Furthermore, with proper authorization, the baby feeding habits data and baby profiles can be shared with others on various social networking platforms, e.g., FACEBOOK™, TWITTER™, YOUTUBE™ and INSTAGRAM™.

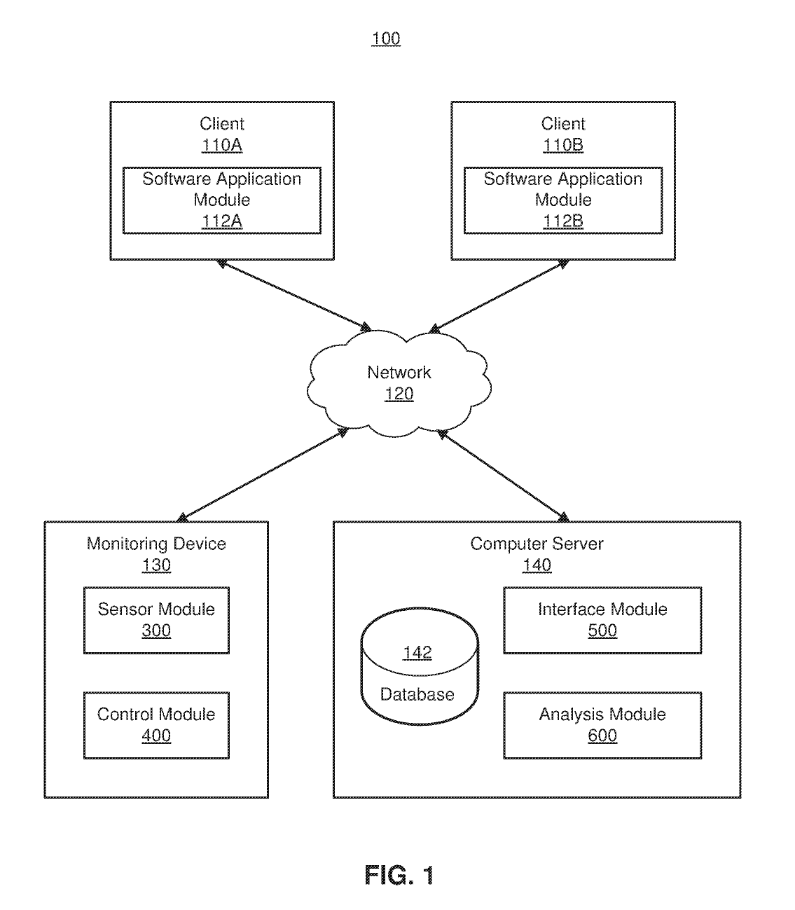

FIG. 1 is a block diagram of a computing environment 100 for monitoring baby feeding habits and for analyzing the baby feeding habits data according to one embodiment. The embodiment illustrated in FIG. 1 includes multiple client devices 110 (e.g., 110A and 110B), a monitoring device 130 and a computer server 140 connected to each other by a network 120. Embodiments of the computing environment 100 can have many client devices 110, monitoring devices 130 and computer servers 140 connected to the network 120. Likewise, the functions performed by the various entities of FIG. 1 may differ in different embodiments.

A client device 110 is an electronic device used by a user to perform functions such as executing software applications, consuming digital content, browsing websites hosted by web servers on the network 120, downloading files, and the like. For example, the client device 110 may be a smart phone, or a tablet, notebook, or desktop computer. The client device 110 includes and/or interfaces with a display device on which the user may view webpages, videos and other content. In addition, the client device 110 provides a user interface (UI), such as physical and/or on-screen buttons, with which the user may interact with the client device 110 to perform functions such as viewing, selecting, and consuming digital content such as webpages, photos, videos and other content.

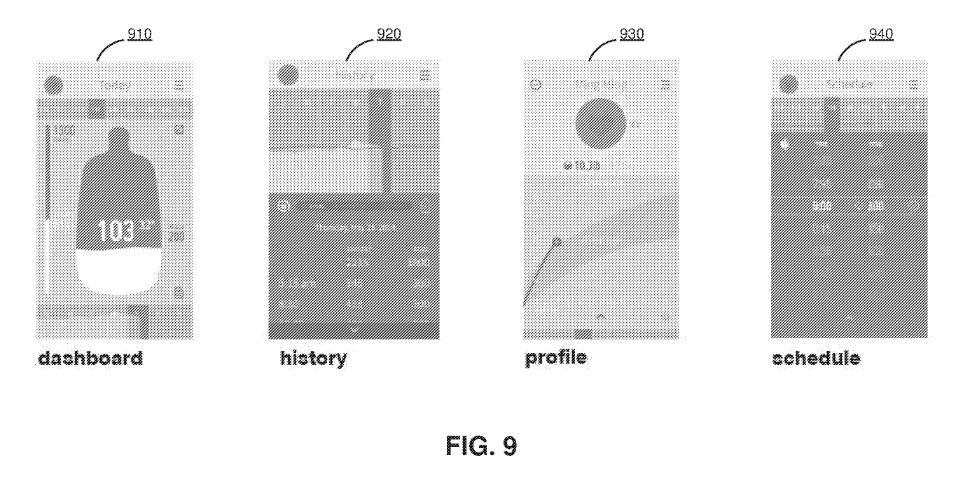

In one embodiment, the client device 110 has a software application module 112 (e.g., 112A for client device 110A and 112B for client device 110B) for executing a software application designed to monitor baby feeding habits anywhere and anytime. For example, once the software application is installed in the client device 110, the software application module 112 communicates with the monitoring device 130 and the computer server 140 to initiate a monitoring process. A caretaker (e.g., a parent or babysitter) prepares baby food with a baby feeding bottle as usual and a user of the client device 110 (e.g., the caretaker herself) can start monitoring the baby food preparation and feeding operations. The baby feeding habits monitoring software application presents a user friendly interface for a user to input baby feeding habits monitoring and feeding information, e.g., temperature of the food, feeding schedule, feed history and baby profile. Responsive to receiving monitoring data from the monitoring device 130 and analysis of the baby feeding habits feeding data from the computer server 140, the baby feeding habits monitoring software application displays the baby feeding habits data and analysis on a display of the client device 110 for the users to digest and share. FIG. 9 through FIG. 15 below further illustrates examples of presenting monitoring and feeding information on the client device 110.

In this disclosure, “baby food” refers any liquid or solid food suitable for young children to consume. Baby food can include, for example, baby formula and liquid such as milk, water and juice. For purposes of simplicity and the description of one embodiment, the baby food will be referred to as milk, but no limitation on the type of baby food that can be analyzed are intended by this terminology. Thus, the operations described herein for monitoring and analyzing baby food consumption can be applied to any type of baby food suitable for young children to consume.

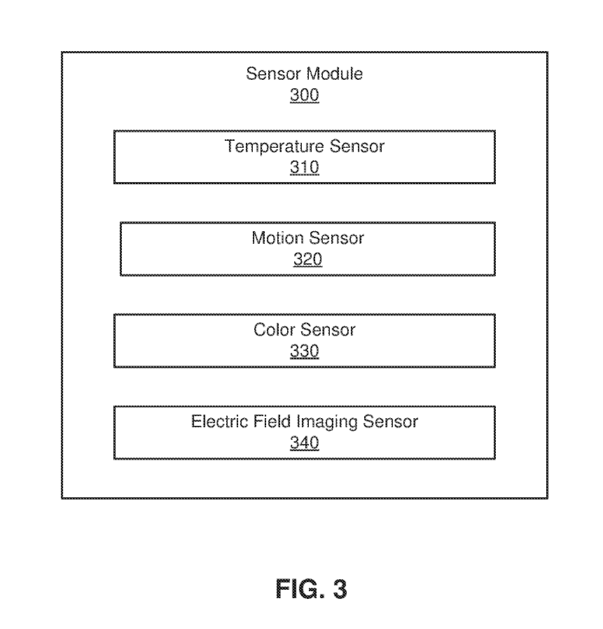

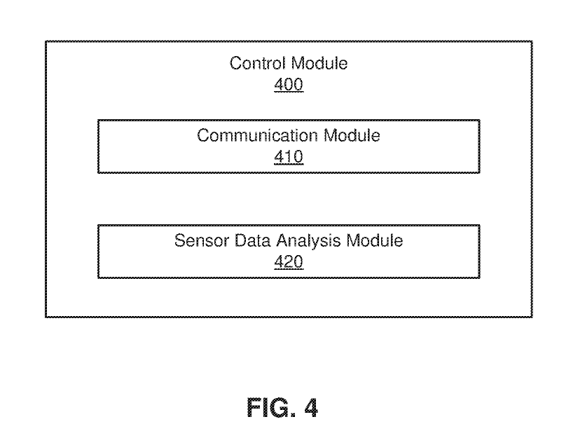

The monitoring device 130 collects baby feeding habits data based on the monitoring of a baby feeding bottle through multiple electronic sensors attached to the baby feeding bottle and derives information about the baby feeding habits, e.g., feeding times, types of baby food (milk, water or juice) and volumes of consumption. In the embodiment illustrated in FIG. 1, the monitoring device 130 includes a sensor module 300 for collecting baby feeding habits data and a control module 400 for deriving information about the baby feeding habits. Other embodiments of the monitoring device 130 can include different and/or additional modules other than the ones shown in FIG. 1. The sensor module 300 is further described with reference to the description of FIG. 3; the control module 400 is further described with reference to the description of FIG. 4.

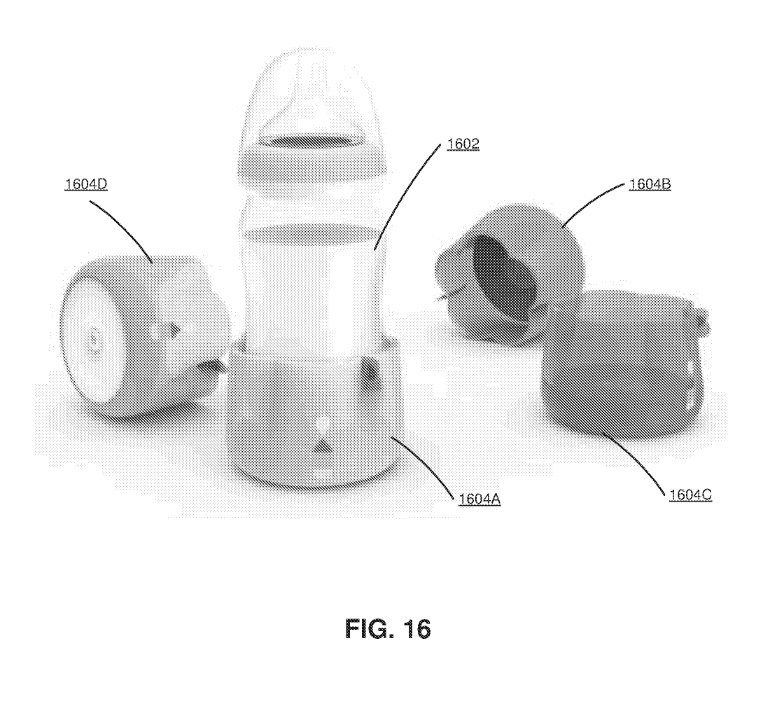

Turning now to FIG. 16, FIG. 16 illustrates an example of a baby feeding bottle 1602 and a monitoring device 1604A attached to the baby feeding bottle 1602 according to one embodiment. In the example shown in FIG. 16, the monitoring device 1604 has a sleeve shape and can be in different translucent colors, e.g., 1604A, 1604B, 1604C and 1604D.

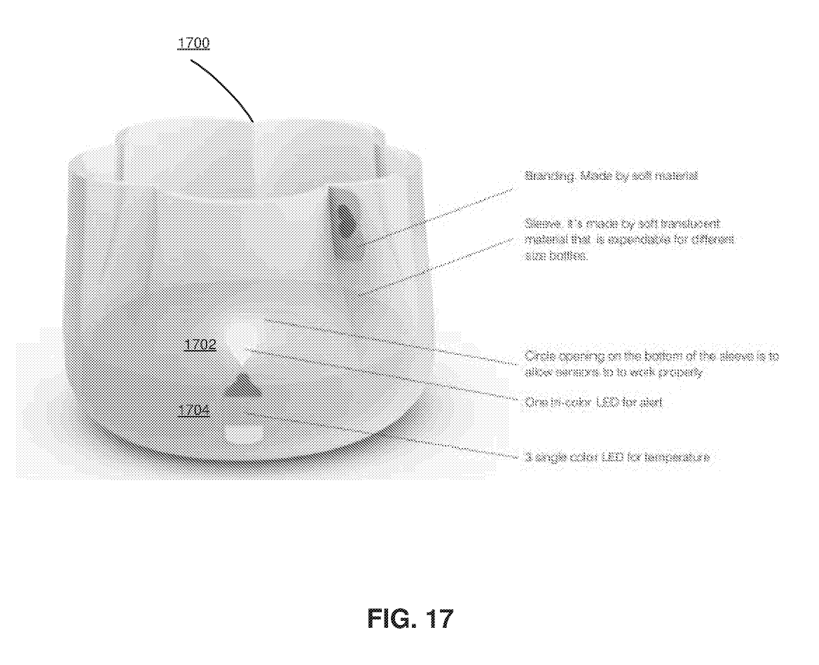

FIG. 17 is an exemplary monitoring device 1700 attached to a baby feeding bottle to collect baby feeding habits data according to one embodiment. The monitoring device 1700 is in a sleeve shape and is made of soft translucent material that is expendable for different sized baby feeding bottles. Various electronic sensors can be attached to the bottom of the monitoring device 1700 to collect baby feeding habits data such as food temperature, type (milk, water or juice), volume and frequency of consumption of the food. The monitoring device 1700 illustrated in FIG. 17 has a light-emitting diode (LED) light indicator 1702 to indicate an alert associated with a baby feeding operation, e.g., time for feeding and expiration of milk in the feeding bottle. The monitoring device 1700 also has a multi-color LED indicator 1704 to indicate the status of milk in the baby bottle in terms of temperature of the milk, e.g., too hot, too cold and proper to drink.

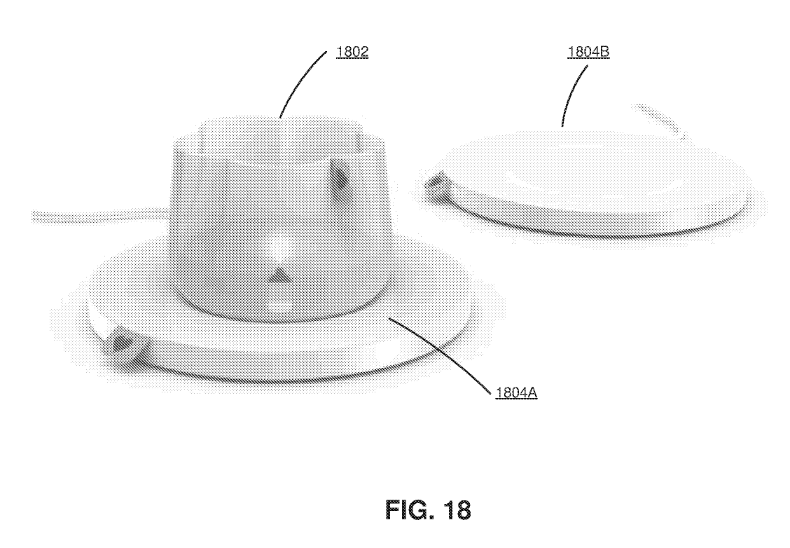

In one embodiment, the monitoring device 130 is safely charged by a wireless charger, e.g., when the baby feeding bottle is empty. FIG. 18 is an exemplary wireless charger 1804A to charge a monitoring device 1802 attached to a baby feeding bottle according to one embodiment. The wireless charger 1804 is made of a plastic disk with a concave dish-shape for the monitoring device 1802 to sit on. The wireless charger 1804 has a micro-USB (Universal Serial Bus) connector to connect the wireless charger 1804 with a power outlet.

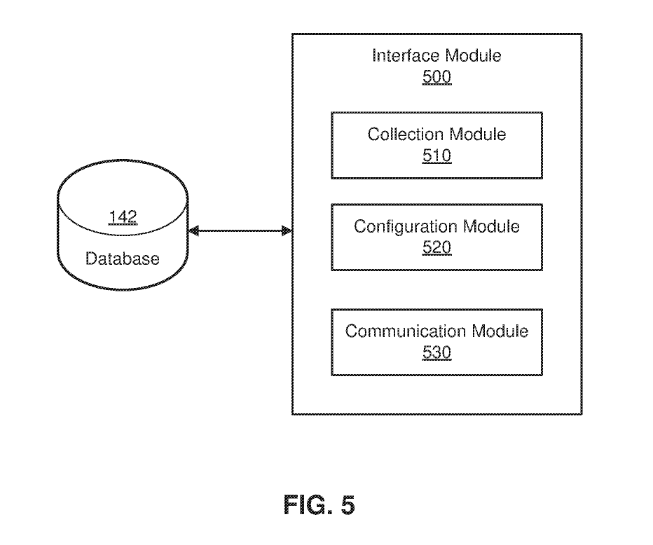

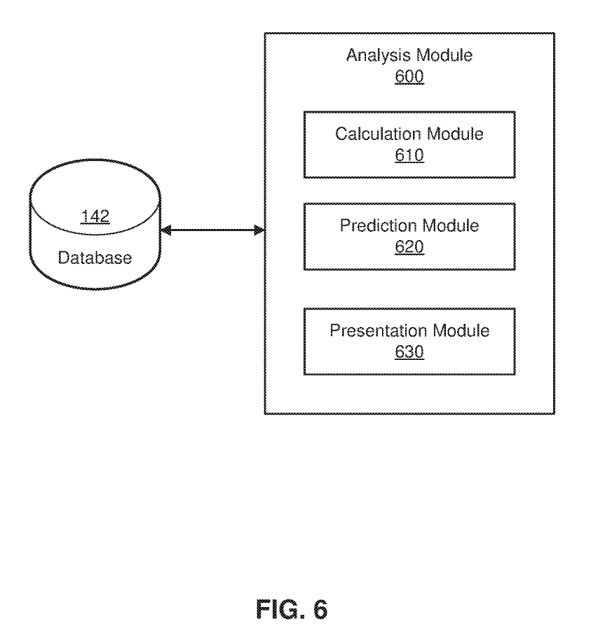

Turning back to FIG. 1, the computer server 140 communicates with the monitoring device 130 and the client device 110 to process the baby feeding habits data collected by the monitoring device 130 and provides the analysis of the baby feeding habits data, alerts and nutritional recommendations based on the analysis to the client device 110 for display. In the embodiment illustrated in FIG. 1, the computer server 140 has a database 142, an interface module 500 and an analysis module 600. The database 142 stores the baby feeding habits data received from the monitoring device 130 and analysis data generated from the baby feeding habits data. The interface module 500 facilitates the communication among the computer server 140, the monitoring device 130 and the client device 110. The analysis module 600 analyzes the baby feeding habits data and generates various analysis data, alerts and recommendations for the users of the client device 110 to display and share. The details of the interface module 500 are further provided with the description of FIG. 5; the details of the analysis module 600 are further provided with the description of FIG. 6.

The network 120 enables communications among the client devices 110, the monitoring device 130 and the computer server 140. In one embodiment, the network 120 comprises the Internet and uses standard communications technologies and/or protocols, e.g., clouding computing. In another embodiment, the entities can use custom and/or dedicated data communications technologies.

### Computing System Architecture

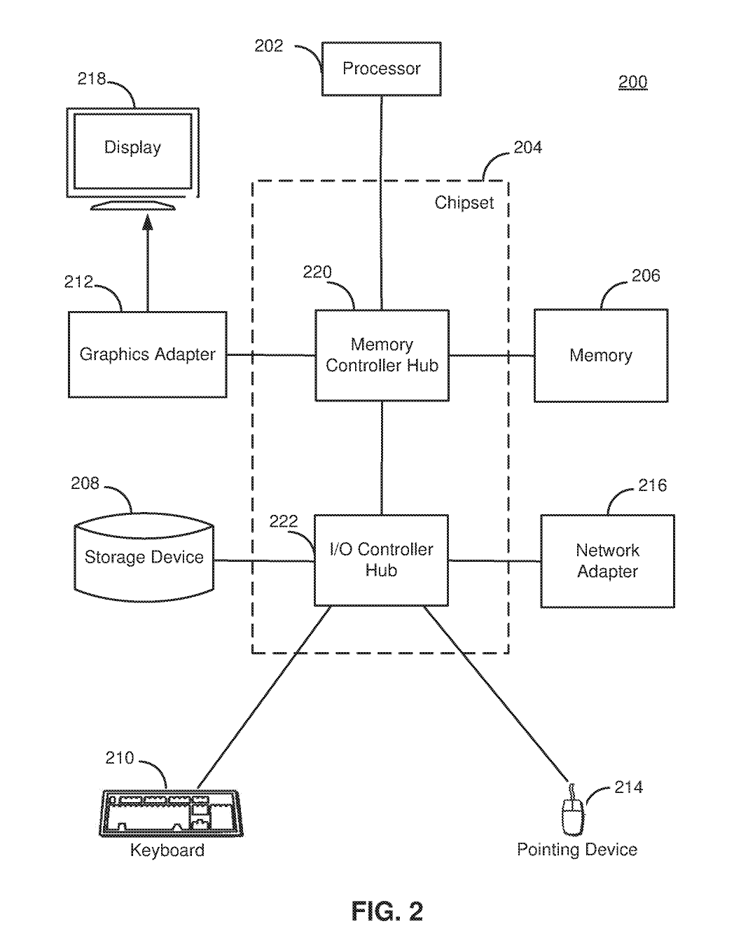

The entities shown in FIG. 1 are implemented using one or more computers. FIG. 2 is a high-level block diagram of a computer 200 for acting as the computer server 140 and/or a client device 110 according to one embodiment. Illustrated are at least one processor 202 coupled to a chipset 204. Also coupled to the chipset 204 are a memory 206, a storage device 208, a keyboard 210, a graphics adapter 212, a pointing device 214, and a network adapter 216. A display 218 is coupled to the graphics adapter 212. In one embodiment, the functionality of the chipset 204 is provided by a memory controller hub 220 and an I/O controller hub 222. In another embodiment, the memory 206 is coupled directly to the processor 202 instead of the chipset 204.

The storage device 208 is any non-transitory computer-readable storage medium, such as a hard drive, compact disk read-only memory (CD-ROM), DVD, or a solid-state memory device. The memory 206 holds instructions and data used by the processor 202. The pointing device 214 may be a mouse, track ball, or other type of pointing device, and is used in combination with the keyboard 210 to input data into the computer system 200. The graphics adapter 212 displays images and other information on the display 218. The network adapter 216 couples the computer system 200 to the network 120.

As is known in the art, a computer 200 can have different and/or other components than those shown in FIG. 2. In addition, the computer 200 can lack certain illustrated components. For example, the computers acting as the computer server 140 can be formed of multiple blade servers linked together into one or more distributed systems and lack components such as keyboards and displays. Moreover, the storage device 208 can be local and/or remote from the computer 200 (such as embodied within a storage area network (SAN)).

As is known in the art, the computer 200 is adapted to execute computer program modules for providing functionality described herein. As used herein, the term “module” refers to computer program logic utilized to provide the specified functionality. Thus, a module can be implemented in hardware, firmware, and/or software. In one embodiment, program modules are stored on the storage device 208, loaded into the memory 206, and executed by the processor 202.

### Baby Feeding Habits Monitoring

The monitoring device 130 attached to a baby feeding bottle collects baby feeding data through a sensor module. FIG. 3 is a block diagram of a control module 300 of the monitoring device 130 configured to collect baby feeding habits data through various electronic sensors according to one embodiment. The control module 300 shown in the embodiment of FIG. 3 includes a temperature sensor 310, a motion sensor 320, a color sensor 330 and an electric field imaging sensor 340. Other embodiments of the control module 300 can include different and/or additional electronic sensors.

The temperature sensor 310 collects temperature data associated with baby food in a baby feeding bottle. In one embodiment, the temperature sensor 310 tracks the changes of temperature of the baby feeding bottle. For example, an empty baby feeding bottle and a baby feeding bottle with milk have different temperatures. It is noted that a baby feeding bottle having different types of baby food, e.g., water and milk, may have different temperatures. The temperature sensor 310 detects the change of temperature of a baby feeding bottle in response to a type of baby food being poured into the bottle and records the change of the temperature. The temperature change data of the baby feeding bottle can be used to determine whether a feeding preparation starts. For example, the temperature change of a baby feeding bottle in response to milk being poured into the empty baby feeding bottle is a reliable indication that a feeding preparation starts.

The temperature sensor 310 continues monitoring the temperature of the baby feeding bottle and provides the temperature data to the control module 400 of the monitoring device 400 and to the computer server 140 for further analysis. The temperature sensor 310 receives the analysis data of the temperature from the control module 400 and/or the computer server 140 and displays an indication on the monitoring device 130 in response to the received analysis data. For example, in response to the temperature data indicating that milk in the baby feeding bottle is not suitable for drinking, e.g., too cold or too hot, the temperature sensor 310 triggers a LED light (e.g., the LED indicator 1704 shown in FIG. 17) to be shown red to alert the baby caretaker.

The motion sensor 320 detects the movement of the baby feeding bottle. It is noted that feeding a baby with a feeding bottle held in proper positions reduces gas accumulated in the baby's stomach. In one embodiment, the motion sensor 320 detects the change of an angle between the baby feeding bottle and a reference point, e.g., an initial position of the baby feeding bottle immediate before the feeding starts. The change of the angle of the baby feeding bottle can help determine whether the feeding is finished. For example, responsive to the baby feeding bottle being upside down indicated by the angle of the feeding bottle, the motion sensor 320 determines that the feeding operation finishes. The motion sensor 320 provides the angle data of the baby feeding bottle to the control module 400 of the monitoring device 130 and the computer server 140 for further analysis, such as determining the volume of the baby food left in the baby feeding bottle.

Given that different baby food may have different colors, the color sensor 330 tracks the change of color of the baby food in a baby feeding bottle. For example, milk is made up of mainly of water with minor amount of solids, such as fat and various proteins (e.g., four types of casein), and appears fairly opaque and white to human eyes. In comparison with milk, water generally appears colorless. In one embodiment, the color sensor 330 uses a set of predefined mappings between commonly consumed baby foods, e.g., milk, water, apple juice, orange juice, popular baby formulas and their corresponding colors to determine the type of food in the baby feeding bottle. For example, the color sensor 330 detects the color of a liquid in the feeding bottle and compares the detected color of the liquid with the colors in the predetermined set mappings. In response to finding a match, the color sensors 330 selects the type of liquid in the predefined mappings with the matched color as the type of liquid in the feeding bottle. The color information tracked by the color sensor 330 is provided to the control module 400 and the computer server 140 for further analysis, e.g., determining the volume of the liquid in the feeding bottle in conjunction with other sensor data and determining whether the liquid in the feeding bottle expires (e.g., too stale to drink).

The electric field imaging sensor 340 tracks proximity data of conductive objects, e.g., milk in the baby feeding bottle, based on the change of an electric field monitored by the electric field imaging sensor 340. In one embodiment, the electric field imaging sensor 340 generates an electric filed by electrical charges and spreads the electric field three-dimensionally around the surface of the baby feeding bottle, which carries the electrical charge. When a conjunctive object, e.g., milk, intrudes the electric field around the surface of the baby feeding bottle, the electric field distribution becomes disturbed. In response to liquid pouring into the baby feeding bottle, the electric field imaging sensor 340 detects distortion of the electric field, e.g., by detecting the change of the electrical charges of the electric field. The proximity data collected by the electric field imaging sensor 340 is provided to the control module 400 and the computer server 140 to determine the volume of the baby food in the baby feeding bottle.

FIG. 4 is a block diagram of a control module 400 of the monitoring device 130 to determine volume of baby food in a baby feeding bottle according to one embodiment. In the embodiment illustrated in FIG. 4, the control module 400 has a communication module 410 and a sensor data analysis module 420. On embodiments of the control module 400 can have different and/or additional modules.

The communication module 410 communicates with the sensor module 300 of the monitoring device 130, the software application module 112 of the client device 110 and the computer server 140. In one embodiment, the communication module 410 receives the baby feeding habits data from the sensor module 300 and provides the feeding habits data to the sensor data analysis module 420 and to the computer server 140 to determine the volume of the baby food in the baby feeding bottle.

The communication module 410 also receives information, such as analysis data and instructions to display various alerts on the monitoring device 130, from the sensor data analysis module 420 and the computer server 140. For example, in response to a determination from the sensor data analysis module 420 or the computer server 140 that the milk in the baby feeding bottle is too hot to drink, the communication module 410 instructs the temperature sensor 310 to power on a LED indicator (e.g., the three-color LED indicator 1704 shown in FIG. 17) to display a warning.

The communication module 410 also receives information from the software application module 112 of the client device 110, such as execution of an instance of the baby feeding monitoring software application on the client device 110. Other information received from the software application module 112 of the client device 110 may include user input, e.g., feeding schedule and feeding preference, from the user of the client device 110. The communication module 410 provides the received information to the sensor data analysis module 420 to guide the analysis of the baby feeding habits data.

The sensor data analysis module 420 receives baby feeding habits data from the sensor module 300 and user preferences regarding the baby feeding preparation and operation from the client device 110 and analyzes the baby feeding habits data. In one embodiment, the sensor data analysis module 420 receives preferred drinking temperature information from the client device 110 and compares the current temperature of the baby food in the feeding bottle with the preferred drinking temperature information. Responsive to the current temperature of the baby food in the feeding bottle being higher than the preferred temperature, the sensor data analysis module 420 determines that the baby food in the feeding bottle is too hot to drink and instructs the communication module 410 to display a warning on the monitoring device 130.

In one embodiment, the sensor data analysis module 420 correlates the baby feeding habits data from various sensors to determine the volume of the baby food in the feeding bottle. For example, the sensor data analysis module 420 determines whether some baby food (e.g., water or milk) has filled the baby feeding bottle based on the data collected by the temperature sensor 310, the motion sensor 320 and the electric field imaging sensor 340. In response to a determination that the baby feeding bottle is filled with the baby food, the sensor data analysis module 420 determines the type of the baby food based on the color data detected by the color sensor 330.

With the knowledge of the type of the baby food, the food temperature, and the angle of the baby feeding bottle, the sensor data analysis module 420 determines the volume of the baby food at the beginning of a feeding operation and at the end of the feeding operation. From the volume data at the beginning and end of the feeding operation, the sensor data analysis module 420 determines the volume of the food consumption and duration for the determined volume of the food consumption.

To reduce “noise” (e.g., false presumption) in the calculating volume data, the sensor data analysis module 420 can adjust the volume data based on additional information associated with the baby feeding habits data. For example, the sensor data analysis module 420 can compare the calculated volume data for different types of baby food, e.g., water, milk and juice, when using the same baby feeding bottle and identifying which factor contributes to the noise of the volume calculation.

The sensor data analysis module 420 may continue monitoring the volume data and other related sensor data, e.g., the temperature of the baby food, even after the end of the feeding operation and use the monitored volume data to determine whether the baby food in the baby feeding bottle has expired, e.g., too stale to drink. For example, responsive to the detection of non-zero amount of milk left in the baby feeding bottle at the room temperature for more than 2 hours, the sensor data analysis module 420 determines that the milk in the feeding bottle is no longer healthy to drink and instructs the communication module 410 to display a warning on the monitoring device 130.

It is noted that the monitoring device 130 is safely charged through a wireless charger such that the various electronic sensors can track the baby feeding habits data. Designed to provide accurate baby feeding habits data and prompt and sufficient warnings, in one embodiment, the sensor data analysis module 420 is configured to calculate light weight volume data, such as volume data for milk only or volume calculation that does not require large computing power. For other types of baby foods or complex volume calculation, the sensor data analysis module 420 requests the computer server 140 for such volume data calculation.

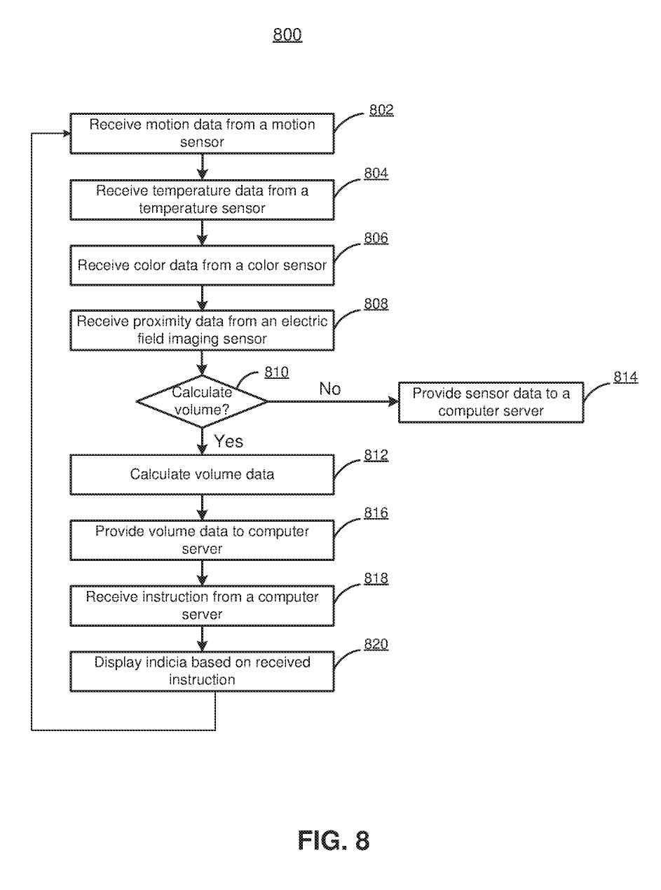

Turning now to FIG. 8, FIG. 8 is an exemplary flowchart illustrating a process for monitoring baby feeding habits data through various electronic sensors according to one embodiment. Initially, the monitoring device 130 receives 802 motion data, e.g., an angle of a baby feeding bottle, from a motion sensor attached to the baby feeding bottle. The angle of the baby feeding bottle helps the monitoring device 130 to determine the status of the feeding operation. For example, an angle representing an upside down position of the baby feeding bottle is likely to indicate that the feeding operation is finished and the monitoring device 130 can calculate how much of milk is left in the feeding bottle.

The monitoring device 130 receives 804 temperature data of the baby food in the feeding bottle from a temperature sensor. The monitoring device 130 can determine whether the baby food is too hot to drink, e.g., by comparing the food temperature with a threshold temperature for the baby food. Responsive to the food temperature exceeding the threshold temperature, the monitoring device 130 displays a warning on the surface of the monitoring device.

The monitoring device 130 receives 806 color information of the baby food in the feeding bottle from a color sensor. Additionally, the monitoring device 130 receives 808 proximity data, e.g., proximity of a conductive object such as milk in the feeding bottle, from an electric field imaging sensor.

Upon receiving the sensor data from various sensors, the monitoring device 130 determines 810 whether calculating the volume of the baby food in the feeding bottle based on the sensor data is light weight, e.g., requiring insignificant computing power that can be sufficiently supported by a wireless charger charging the monitoring device 130. Responsive to a determination that the volume calculation is light weight, the monitoring device 130 calculates 812 the volume and provides 816 the calculated volume data to a computer server e.g., the computer sever 140 illustrated in FIG. 1; responsive to a determination that the volume calculation is not light weight, the monitoring device 130 provides 814 the sensor data to the computer server 140 for further processing.

The monitoring device 130 receives 818 instructions from the computer server 140 and displays 820 an indicia based on the received instructions, e.g., a red light to indicate that the milk is too hot to drink, or an alert to indicate that it is time to feed the baby according to a feeding schedule. The monitoring device 130 repeats the steps of 802 to 820 in real time and continuously as long as the baby feeding bottle is used to feed a baby.

### Baby Feeding Habits Data Analysis

In addition to the analysis of the baby feeding habits data by the sensor data analysis module 420 at the monitoring device 130, the computer server 140 can also provide real time and in-depth analysis of the baby feeding habits data. In one embodiment, the computer server 140 has an interface module 500 for communicating with the monitoring device 130 and client devices 110, and an analysis module 600 for in-depth analysis of the baby feeding habits data.

FIG. 5 is a block diagram of an interface module 500 of the computer server 140 to communicate with client devices 110 and monitoring devices, e.g., the monitoring device 130 in FIG. 1, according to one embodiment. In the embodiment shown in FIG. 5, the interface module 500 has a collection module 510, a configuration module 520 and a is communication module 530. Other embodiments of the interface module 500 can have different and/or additional modules.

The collection module 510 interacts with the client devices 110 to receive user input to the baby feeding habits monitoring application executed on the client devices 110. Examples of user input include feeding schedule, feeding preparation instructions and demographic information (e.g., age, gender and race) and photos to set up a baby profile. In one embodiment, the collection module 510 stores the received user input in the database 142. In another embodiment, the collection module 510 provides the received user input to the communication module 530 for further processing, e.g., instructing the monitoring device to display an alert for incoming feeding time according to the feeding schedule.

The configuration module 520 monitors the operation of the baby feeding habits monitoring application executed on the client devices 110 and prepares sharing online of baby feeding habits data and baby profile selected by the users of the baby feeding habits monitoring software application. In one embodiment, the configuration module 520 provides any software updates, such as feature updates and security patches, to the software application module 112 of the client device 110 for smooth and secure operation of the baby feeding habits monitoring software application. The configuration module 520 controls and monitors a website, where the users of the baby feeding habits monitoring software application can download software updates and benchmark data related to healthy baby feeding and nutritional intakes. In one embodiment, the configuration module 520 includes links to various social networking platforms, e.g., e.g., FACEBOOK™, TWITTER™, YOUTUBE™ and INSTAGRAM™, where the users of the baby feeding habits monitoring application can share their baby feeding habits data.

The communication module 530 communicates with the client devices 110 and the monitoring device 130. In one embodiment, the communication module 530 provides the received user input on feeding schedule, feeding preference, feeding preparation instructions, and the like, to the monitoring device 130. Based on the user input, the monitoring device 130 displays various alerts, e.g., an alert in response to the milk in the feeding bottle being too hot to drink.

The communication module 530 receives the baby feeding habits data collected by the sensors of the monitoring device 130 and provides the received sensor data to the analysis module 600 of the computer server 140 to calculate volume of the baby food in the feeding bottle and other statistics based on the received sensor data.

In another embodiment, the communication module 530 receives from the analysis module 600 of the computer server 140 analysis of the baby feeding habits data, nutritional recommendations/tips and instructions on how to present the analysis data in a user friendly way, and provides the received information to the software application module 112 of the client device 110 for display.

The analysis module 600 of the computer server 140 calculates volume of the baby food in the baby feeding bottle and other statistics based on the baby feeding habits data collected by the monitoring device 130. FIG. 6 is a block diagram of the analysis module 600 to analyze baby feeding habits data according to one embodiment. The analysis module 600 illustrated in FIG. 6 has a calculation module 610, a prediction module 620 and a presentation module 630. Other embodiments of the analysis module 600 can have different and/or additional modules.

The calculation module 610 calculates the volume of baby food in a baby feeding bottle based on the baby feeding habits data collected by the sensors of the monitoring device 130. In one embodiment, in response to an indication that the baby feeding bottle is filled with some baby food, the calculation module 610 determines the type of the baby food based on the color data detected by the color sensor 330.

Given the knowledge of the type of the baby food, the food temperature, and the angle of the baby feeding bottle, the calculation module 610 calculates the volume of the baby food in the feed bottle and recalculates the volume of the baby food during the feeding operation. From the volume data for the beginning and end of the feeding operation, the calculation module 610 determines the amount of baby food consumed by the baby. Responsive to a detection of non-zero amount of milk or juice left in the baby feeding bottle at the room temperature for more than a predetermined period of time, e.g., 2 hours, the calculation module 610 determines that the milk/juice in the feeding bottle is no longer healthy to drink and generates a warning for the monitoring device 130 to display. The calculation module 610 provides the volume data in real time to the communication module 530 of the computer server 140, which provides the calculated volume data to the client device 110 for display.

The prediction module 620 receives baby feeding habits data from multiple monitoring devices 130 supported by the computer server 140 and correlates the received baby feeding habits data. In one embodiment, the prediction module 620 correlates the received baby feeding habits data by classifying the received baby feeding habits data into different classes based on one or more classification criteria, e.g., age, race, gender, weight, height, types of baby food and feeding time. In another embodiment, the prediction module 620 correlates the received baby feeding data with benchmark data, e.g., National Survey of

Maternity Practices in Infant Nutrition and Care published by U.S. Centers for Disease Control and Prevention (CDC), by comparing the collected baby feeding habits data with the CDC benchmark data. Based on the correlation, the prediction module 620 generates summaries of the received baby feeding habits data, predictions of trends and nutritional recommendations for healthy baby feeding habits.

The presentation module 630 generates instructions on how to present the analysis data of the baby feeding habits data from the calculation module 600 and the prediction module 620 and provides the presentation instructions associated with the analysis data to the client device 110.

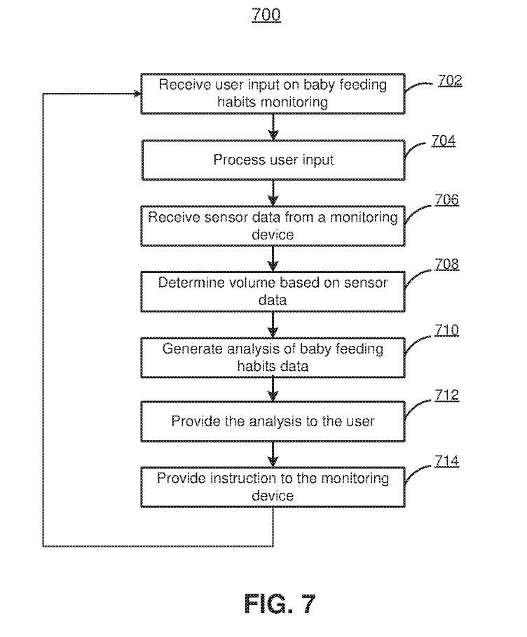

FIG. 7 is an exemplary flowchart illustrating a process for analyzing baby feeding habits data by a computer server 140 according to one embodiment. Initially, the computer sever 140 receives 702 user input on baby feeding habits monitoring, e.g., feeding schedule, appropriate temperature of the baby food for consumption, instructions for preparing the baby food. The computer server 140 processes 704 the user input, e.g., storing the user input in a computer storage at the computer server 140 or providing the user input to the monitoring device 130 to guide the baby feeding habits monitoring.

The computer server 140 receives 706 sensor data from the monitoring device 130 attached to the baby feeding bottle. The received sensor data describes various aspects of the baby feeding operation monitored by the monitoring device 130 such as the temperature of the baby food in the feeding bottle, the color of the baby food, the angle of the baby feeding bottle and proximity of the baby food in the feeding bottle. Based on the received sensor data, the computer server 140 calculates 708 the volume of the baby food in the baby feeding bottle and generates 710 various types of analysis of the sensor data, predictions and nutritional recommendations. The computer sever 140 provides 712 the analysis, predictions and nutritional recommendations to the users of the client device 110 for display. The computer server 140 generates instructions, e.g., alerts for next feeding time according to a user selected feeding schedule, and provides 714 instructions to the monitoring device 130. The computer server 140 repeats the steps of 702 to 714 in response to user input from client devices 110 and/or baby feeding habits data received from monitor devices 130 supported by the computer server 140.

### Presenting Baby Feeding Habits Data and Analysis

In addition to enable users of the baby feeding habits monitoring application to monitor and record their babies' feeding times and intakes regardless where the users are, the computer server 140 presents the baby feeding habits data, nutritional recommendations and benchmark comparisons in a user friendly way on the users' computing devices, e.g., users' smart phones. FIG. 9 through FIG. 15 illustrate examples of presenting baby feeding habits data, nutritional recommendations and benchmark comparisons in a user friendly way on a user's smart phone.

FIG. 9 illustrates examples of graphical user interface to present various types of information associated with baby feeding habits data on a smart phone, according to one embodiment. The examples shown in FIG. 9 include a dashboard screen 910, a history data screen 920, a baby profile screen 930 and a feeding schedule screen 940. The dashboard screen 910 represents a current status of a baby feeding operation. The history data screen 920 shows a page of historical baby feeding habits data. The profile screen 930 shows a profile of a baby whose feeding habits are monitored by the monitoring device 130 and the computer server 140. The feeding schedule screen 940 captures a user selected feeding schedule in 15-minute intervals.

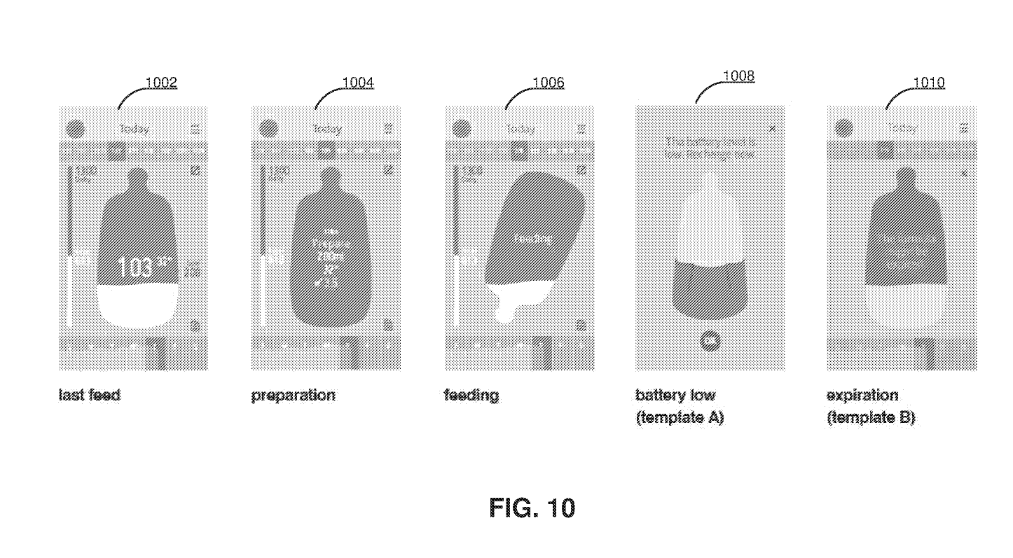

FIG. 10 illustrates examples of a graphical representation of a dashboard for presenting baby feeding habits data on a smart phone according to one embodiment. The examples of the graphical representation of the dashboard show various stages of a baby feeding operation. For example, the dashboard 1002 shows the baby feeding habits data after the last feed, including the temperature (e.g., 103 F. or 32° C.) of the baby food in the feeding bottle, the target of amount of intake (e.g., 200 ml), the amount of actual consumption (e.g., 613 ml up to the last feed). The dashboard 1004 shows a snippet of the feeding preparation according to a user selected feeding preference, e.g., the amount (200 ml) and appropriate drinking temperature (32° C.). The dashboard 1006 indicates a baby being fed, where the feeding bottle is turned upside down and a text message (“feeding”) indicating the feeding action is being performed by a caretaker. The dashboard 1008 shows a warning message for low battery of a charger (e.g., the wireless charger 1804A in FIG. 18), which charges the monitoring device 130. The dashboard 1010 shows a warning message indicates that the baby formula in the feeding bottle may be expired, e.g., the baby formula left in the feeding bottle at the room temperature for more than 2 hours.

In addition to the examples shown in FIG. 10, the baby feeding habits monitoring application can integrate with other components of the client device 110 to further improve the usability and functionality of the baby feeding habits monitoring application. For example, the baby feeding habits monitoring application on a smart phone uses the smart phone's accelerometer to animate the flow of milk in the feeding bottle by tilting the smart phone. In another example as shown in the dashboard 1010, the baby feeding habits monitoring application uses a yellowish color to represent the stale baby formula in the feeding bottle, in contrast of a white translucent color representing fresh baby formula in the feeding bottle.

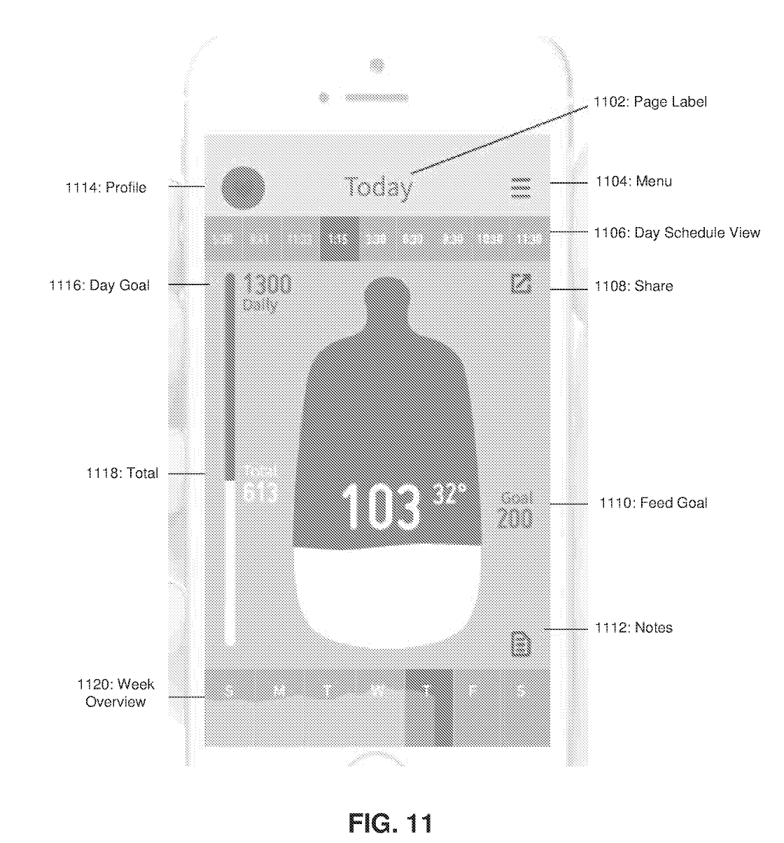

FIG. 11 is an exemplary graphical representation of a dashboard showing various information of baby feeding habits data on a smart phone according to one embodiment. The dashboard shows a page label 1102 to indicate the date of the baby feeding habits data, a menu 1104 to navigate through the baby feeding habits data by swiping or tapping the menu symbol 1104, a feeding goal 1110 set by the user (e.g., 200 ml) and notes 1112 for viewing a list of notes written by the user. The heart shape symbol 1114 represents a profile of the baby whose feeding habits are monitored. Users can slide to switch, tap or pull the heart symbol to view and edit the profile. The vertical bar on the left of the dashboard shows the current total amount of consumption 1118 (e.g., 613 ml) over a goal of daily total consumption 1116 (e.g., 1300 ml) set for the day. In addition to a daily view as shown in the dashboard, the user can selects a weekly view 1120, where selecting a specific date displays historical baby feeding habits data associated with the selected date.

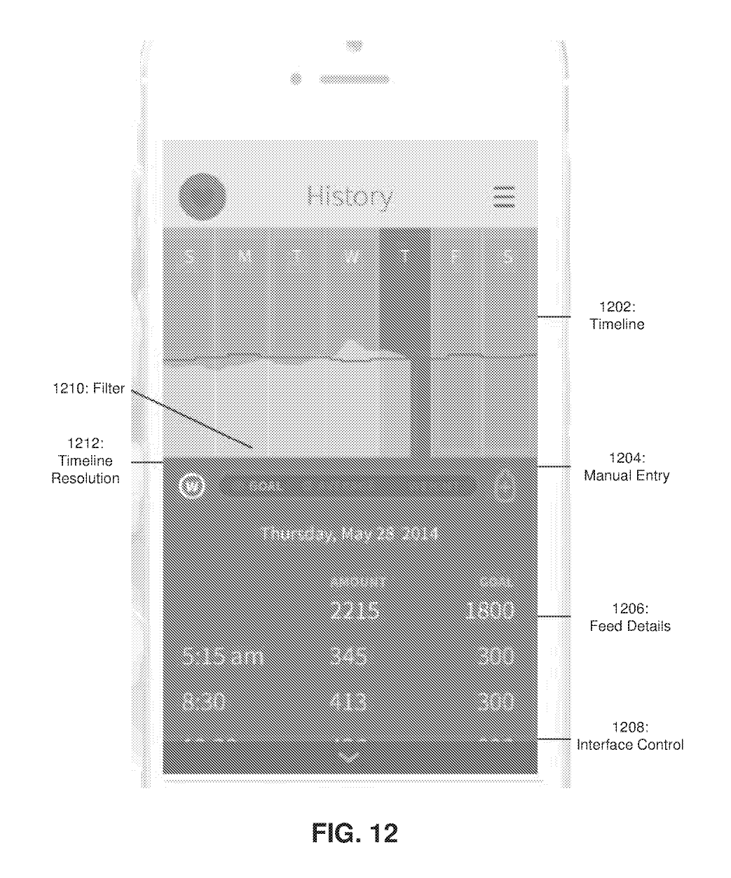

FIG. 12 illustrates an exemplary graphical user interface to present historical baby feeding habits data at a smart phone according to one embodiment. The timeline 1202 enables a user to view the baby's drinking trends over a particular day of interest to the user. The manual entry symbol 1204 opens an overlay for a user to manually edit feed amount and scheduling. Feed details portion 1206 in the dashboard shows details for each baby feeding operation, e.g., timing, actual amount of consumption, goal (i.e., target amount of consumption) and timing. The user can scroll up and down to view more feeding details; the feeding details in the example are arranged from the most recent at the top to the earliest at the bottom. The filter 1210 enables a user to select a benchmark, daily consumption goal, peer average consumption and weight, for comparing the baby's feeding habits data with the selected benchmark. The user can turn off the filter option. The timeline resolution 1212 enables a user to toggle between weekly and monthly view of baby feeding habits data.

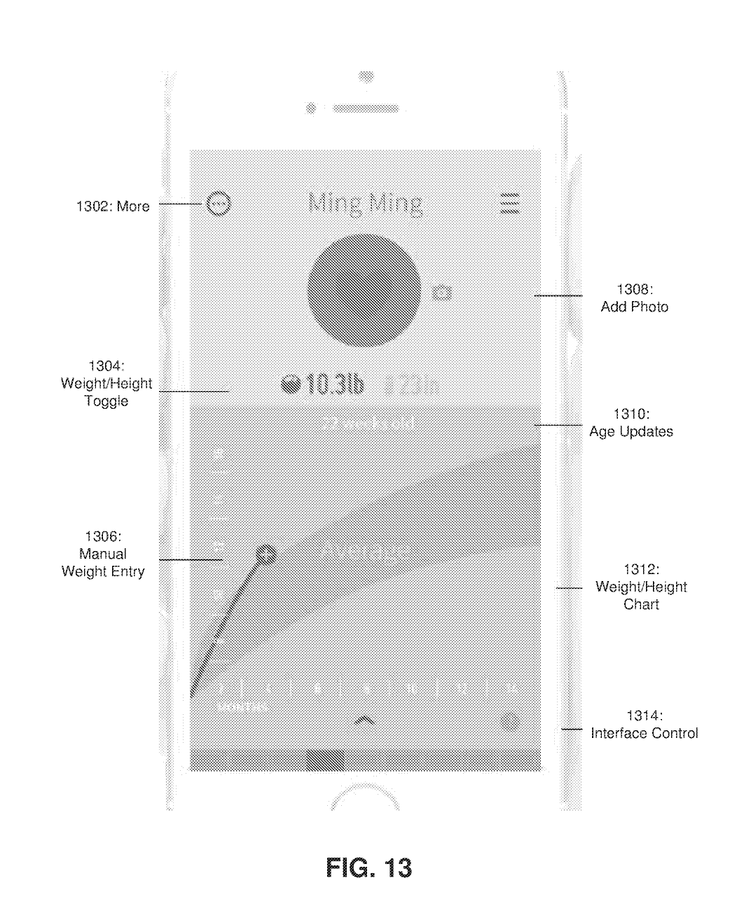

FIG. 13 illustrates an exemplary graphical user interface to present profile of a baby named “Ming Ming” on a smart phone, whose feeding habits data are collected and analyzed, according to one embodiment. A user can add 1308 a photo of Ming Ming by clicking the plus sign, which provides an access to a photo library at the smart phone. The user can update Ming Ming's personal information, e.g., age (1310), weight (1306), or toggle (1304) between weight and height data. The user can view Ming Ming's weight/height chart 1312, which can be zoomed in and zoomed out to view daily and/or weekly weight/height data. Clicking the “More” symbol 1302 enables the user to edit profile, set preferences, invite others to view the profile. The user can close the profile page by tapping the interface (1314) at the bottom of the profile page.

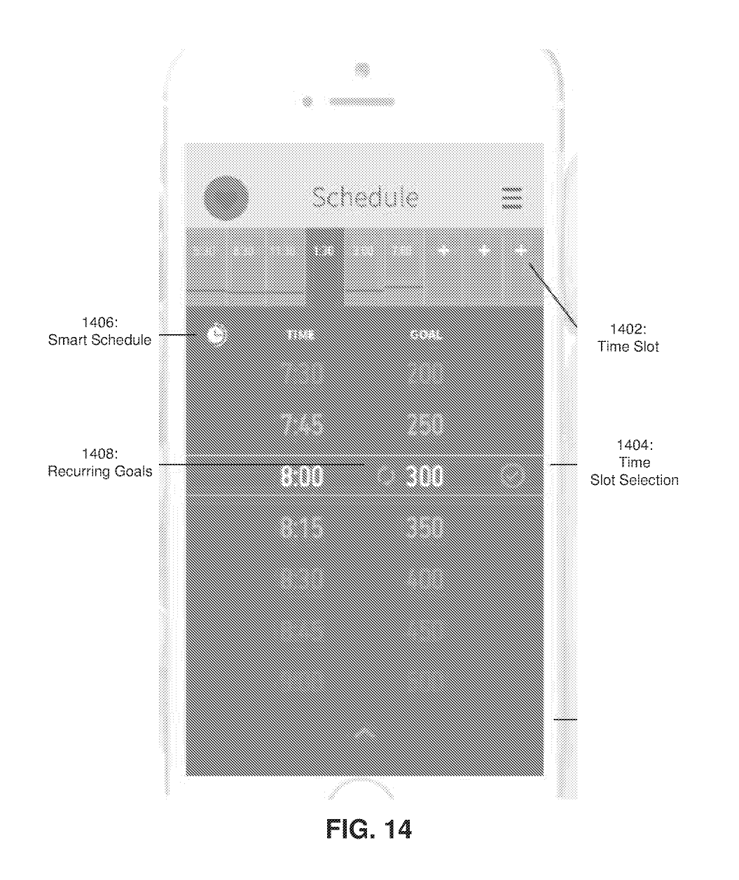

FIG. 14 illustrates an exemplary graphical user interface to present feeding scheduling information on a smart phone according to one embodiment. Tapping the time slot symbol 1402 allows a user to select a time slot or add a new one. Selecting a time slot 1404 shows the timing and target amount of consumption for the selected time slot. Tapping the check symbol allows the user to confirm the selection. The smart schedule symbol 1406 allows the user to revert back to the last feeding schedule set by the user. Editing the recurring goals symbol 1408 allows the user to set the target amount of consumption across multiple time slots.

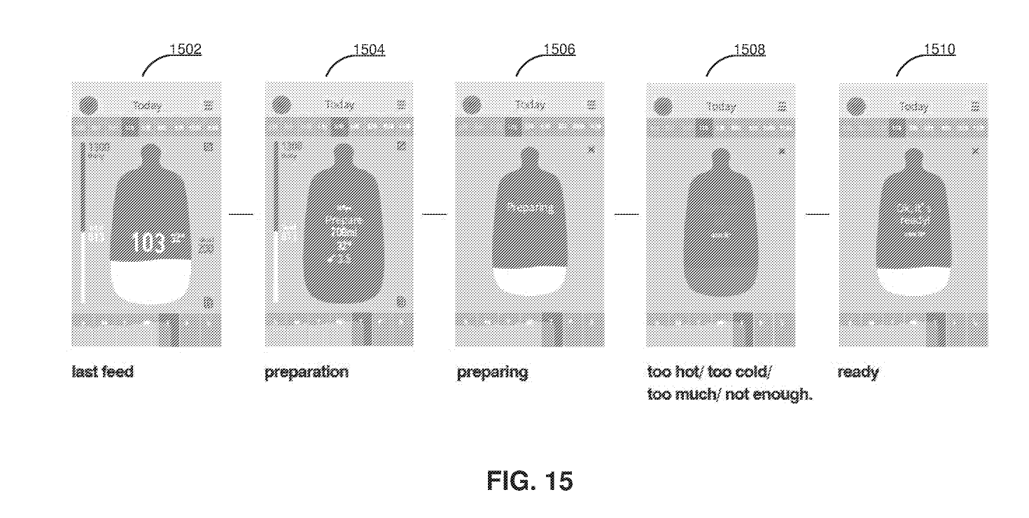

FIG. 15 illustrates an exemplary graphical user interface to display baby feeding status and alerts on a smart phone according to one embodiment. The example shown in FIG. 15 shows five states of the baby feeding operation: last feed 1502, start of preparation 1504, in the process of the preparation 1506, warning 1508 (e.g., too hot/cold, and too much/not enough) and ready to consume 1510. In each state, baby feeding related information is shown on a display of the smart phone. For example, at the start of preparation 1504, the feeding preference, e.g., total amount (200 ml), temperature (32° C.) and amount of baby formula needed (3.5 spoons), is provided to guide the preparation. If the temperature of the content in the feeding bottle is too hot to consume, e.g., 45° C. against 32° C. goal, a warning message is shown to the user. When the content is ready to consume, a friendly message, e.g., “Ok, it's ready!” is displayed to the user.

### General

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the invention may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

1. A computer-implemented method for monitoring baby feeding habits, comprising:
receiving a plurality of sensor data from a plurality of electronic sensors included in a monitoring device, the plurality of sensor data describing a plurality aspects of baby feeding habits monitored by the monitoring device;
generating an analysis of the plurality of sensor data;
generating a plurality of instructions on presenting the analysis on a client device different than the monitoring device; and
providing the generated analysis and the plurality of instructions to the client device for display.

2. The method of claim 1, wherein the plurality of sensor data comprises:
temperature data of a baby feeding bottle monitored by the monitoring device;
motion data describing an angle of the baby feeding bottle during a feeding operation;
color data describing color of content in the baby feeding bottle; and
proximity data describing proximity of the content in the baby feeding bottle.

3. The method of claim 2, wherein the plurality of the electronic sensors comprises:
a temperature sensor for detecting the temperature data;
a motion sensor for detecting the motion data;
a color sensor for detecting the color data; and
an electric field imaging sensor for detecting the proximity data.

4. The method of claim 1, wherein generating the analysis of the plurality of sensor data comprises:
calculating volume of content in a baby feeding bottle based on the plurality of sensor data.

5. The method of claim 4, wherein calculating the volume of content in the baby feeding bottle comprises:
detecting start of a baby feeding operation based on at least one of temperature of the baby feeding bottle, an angle of the baby feeding bottle, and proximity of the content in the baby feeding bottle;
detecting a type of food of the content in the baby feeding bottle based on color of the content; and
responsive to detecting the start of the baby feeding operation, calculating the volume of the content in the baby feeding bottle based at least in part on the type of food of the content.

6. The method of claim 1, wherein generating an analysis of the plurality of sensor data further comprises:
detecting whether there is content in a baby feeding bottle;
detecting a duration of time during which the content has been present in the baby feeding bottle and temperature of the baby feeding bottle; and
responsive to determining that the temperature is at least at a predefined temperature and that the duration of time is greater than a predefined period of time, determining that the content in the baby feeding bottle is not healthy for consumption.

7. The method of claim 1, wherein generating the analysis of the plurality of sensor data further comprises:
receiving another plurality of sensor data from a plurality of other monitoring devices, each of the plurality of other monitoring devices monitoring feeding habits of other babies;
correlating the plurality of sensor data with the other plurality of sensor data; and
generating a summary of the baby feeding habits based on the correlation.

8. A non-transitory computer readable storage medium storing executable computer program instructions for monitoring baby feeding habits, the instructions when executed by a computer processor cause the computer processor to:
receive a plurality of sensor data from a plurality of electronic sensors included in a monitoring device, the plurality of sensor data describing a plurality aspects of baby feeding habits monitored by the monitoring device;
generate an analysis of the plurality of sensor data;
generate a plurality of instructions on presenting the analysis on a client device different than the monitoring device; and
provide the generated analysis and the plurality of instructions to the client device for display.

9. The computer readable storage medium of claim 8, wherein the plurality of sensor data comprises:
temperature data of a baby feeding bottle monitored by the monitoring device;
motion data describing an angle of the baby feeding bottle during a feeding operation;
color data describing color of content in the baby feeding bottle; and
proximity data describing proximity of the content in the baby feeding bottle.

10. The computer readable storage medium of claim 9, wherein the plurality of the electronic sensors comprises:
a temperature sensor for detecting the temperature data;
a motion sensor for detecting the motion data;
a color sensor for detecting the color data; and
an electric field imaging sensor for detecting the proximity data.

11. The computer readable storage medium of claim 8, wherein generating the analysis of the plurality of sensor data comprises:
calculate volume of content in a baby feeding bottle based on the plurality of sensor data.

12. The computer readable storage medium of claim 11, wherein calculating the volume of content in the baby feeding bottle comprises:
detect start of a baby feeding operation based on at least one of temperature of the baby feeding bottle, an angle of the baby feeding bottle and proximity of the content in the baby feeding bottle;
detect a type of food of the content in the baby feeding bottle based on color of the content;
responsive to a detecting the start of the baby feeding operation, calculate the volume of the content in the baby feeding bottle based at least in part on the type of food of the content.

13. The computer readable storage medium of claim 8, wherein generating the analysis of the plurality of sensor data further comprises:
detect whether there is content in a baby feeding bottle;
detect a duration of time during which the content has been present in the baby feeding bottle and temperature of the baby feeding bottle; and
responsive to determining that the temperature is at least at a predefined temperature and that the duration of time is greater than a predefined period of time, determine that the content in the baby feeding bottle is not healthy for consumption.

14. The computer readable storage medium of claim 8, wherein generating the analysis of the plurality of sensor data further comprises:
receive another plurality of sensor data from a plurality of other monitoring devices, each of the plurality of other monitoring devices monitoring feeding habits of other babies;
correlate the plurality of sensor data with the other plurality of sensor data; and
generate a summary of the baby feeding habits based on the correlation.

15. A device for monitoring baby feeding habits, comprising:
a plurality of electronic sensors surrounding a baby feeding bottle;
a computer processor for executing computer program instructions; and
a non-transitory computer readable storage device storing computer program instructions executable to perform steps comprising:
receiving a plurality of sensor signals detected by the plurality of electronic sensors;
analyzing the plurality of sensor signals to generate a plurality of sensor data describing a plurality of aspects of the baby feeding habits; and
providing the generated plurality of sensor data to a computer server, the computer server configured to analyze the plurality of sensor data in real time and provide an analysis of the plurality of sensor data for display to a client device different than the device for monitoring baby feeding habits.

16. The device of claim 15, wherein the computer program instructions for analyzing the plurality of sensor signals are further executed by the computer processor to perform steps of:
generating temperature data of the baby feeding bottle from temperature sensor signals;
generating motion data describing an angle of the baby feeding bottle during a feeding operation from motion sensor signals;
generating color data describing color of content in the baby feeding bottle from color sensor signals; and
generating proximity data describing proximity of the content in the baby feeding bottle from electronic field imaging sensor signals.

17. The device of claim 15, wherein the computer program instructions for analyzing the plurality of sensor signals are further executed by the computer processor to perform steps of:
determining whether calculating volume of content in the baby feeding bottle requires computing power greater than a predefined threshold;
responsive to determining that calculating the volume of the content requires computing power larger greater than the predefined threshold, requesting the computer server to compute the volume of the content; and
responsive to determining that calculating the volume of the content does not require computing power greater than the predefined threshold, calculating, by the device, the volume of the content in the baby feeding bottle.

18. The device of claim 17, wherein calculating the volume of the content in the baby feeding bottle comprises:
detecting start of a baby feeding operation based on at least one of temperature of the baby feeding bottle, an angle of the baby feeding bottle, and proximity of the content in the baby feeding bottle;
detecting a type of food of the content in the baby feeding bottle based on color of the content; and
responsive to detecting the start of the baby feeding operation, calculating the volume of the content in the baby feeding bottle based at least in part on the type of food of the content.

19. The device of claim 15, wherein the computer program instructions are further executed by the computer processor to perform steps of:
receiving, from the computer server, instructions associated with the monitored baby feeding habits; and
displaying an indicia corresponding to the received instructions on a display of the client device.

20. The device of claim 19, wherein the computer program instructions are further executed by the computer processor to perform steps of:
displaying a first indicia indicating that content in the baby feeding bottle is too hot for consumption;
displaying a second indicia indicating that the content in the baby feeding bottle is no longer healthy for consumption; and
displaying an alert indicating that it is time to start a feeding operation according to a preferred feeding schedule.
